SEQUENCE LISTING (16 sequences)

SEQ ID NO: 1 - protein, 22 aa, Artificial Sequence
  Synthesized from human ribosomal P protein
KKEEKKEESEEEDEDMGFGLFD

SEQ ID NO: 2 - protein, 20 aa, Artificial Sequence
  Synthesized from human ribosomal P protein
LIKAAGVNVEPFWPGLFAKA

SEQ ID NO: 3 - protein, 10 aa, Artificial Sequence
  Synthesized from human ribosomal P protein
LIKAAGVNVE

SEQ ID NO: 4 - protein, 11 aa, Artificial Sequence
  Synthesized from human ribosomal P protein
GVNVEPFWPGL

SEQ ID NO: 5 - protein, 10 aa, Artificial Sequence
  Synthesized from human ribosomal P protein
PFWPGLFAKA

SEQ ID NO: 6 - protein, 80 aa, Artificial Sequence
  Synthesized from human ribosomal P protein
LIKAAGVNVEPFWPGLFAKALIKAAGVNVEPFWPGLFAKALIKAAGVNVEPFWPGLFAKALIKAAGVNVEPFWPGLFAKA

SEQ ID NO: 7 - protein, 40 aa, Artificial Sequence
  Synthesized from human ribosomal P protein
LIKAAGVNVELIKAAGVNVELIKAAGVNVELIKAAGVNVE

SEQ ID NO: 8 - protein, 44 aa, Artificial Sequence
  Synthesized from human ribosomal P protein
GVNVEPFWPGLGVNVEPFWPGLGVNVEPFWPGLGVNVEPFWPGL

SEQ ID NO: 9 - protein, 40 aa, Artificial Sequence
  Synthesized from human ribosomal P protein
PFWPGLFAKAPFWPGLFAKAPFWPGLFAKAPFWPGLFAKA

SEQ ID NO: 10 - protein, 13 aa, Artificial Sequence
  Synthesized from human ribosomal P protein
EEEDEDMGFGLFD

SEQ ID NO: 11 - protein, 52 aa, Artificial Sequence
  Synthesized from human ribosomal P protein
EEEDEDMGFGLFDEEEDEDMGFGLFDEEEDEDMGFGLFDEEEDEDMGFGLFD

SEQ ID NO: 12 - protein, 3 aa, Artificial Sequence
  Synthesized from human ribosomal P protein
KKK

SEQ ID NO: 13 - protein, 3 aa, Artificial Sequence
  Synthesized from human ribosomal P protein
KKK

SEQ ID NO: 14 - protein, 3 aa, Artificial Sequence
  Synthesized from human ribosomal P protein
KKK

SEQ ID NO: 15 - protein, 3 aa, Artificial Sequence
  Synthesized from human ribosomal P protein
KKK

SEQ ID NO: 16 - protein, 3 aa, Artificial Sequence
  Synthesized from human ribosomal P protein
KKK

(19) United States
(12) Patent Application Publication
(10) Pub. No.: US 2011/0028409 A1
(43) Pub. Date: Feb. 3, 2011

(54) DETECTION OF ANTI-RIBOSOMAL P PROTEIN ANTIBODIES BY MEANS OF SYNTHETIC PEPTIDES

(75) Inventors: Federico PRATESI, Siena (IT); Maria Claudia ALCARO, Siena (IT); Mario CHELLI, Siena (IT); Paolo FANTINI, Siena (IT); Francesco LOLLI, Siena (IT); Ilaria PAOLINl, Siena (IT); Anna Maria PAPINI, Siena (IT); Paolo ROVERO, Siena (IT); Paola MIGLIORINI, Siena (IT)

Correspondence Address: LUCAS & MERCANTI, LLP, 475 PARK AVENUE SOUTH, 15TH FLOOR, NEW YORK, NY, 10016, US

(73) Assignee: TOSCANA BIOMARKERS S.R.L., SIENA (IT)

(21) Appl. No.: 12/842,197

(22) Filed: Jul. 23, 2010

(30) Foreign Application Priority Data
Jul. 31, 2009 (EP) 09167004.2

(51) Int. Cl. A61K 38/16; C07K 14/00; G01N 33/53; A61P 37/00

(52) U.S. Cl. 514/21.3; 530/324; 435/7.92

(57) ABSTRACT

The present invention refers peptides and methods for the identification of diagnostic antibodies in auto-immune diseases such as Systemic Lupus Erythematosus (SLE), and therefore useful tools for diagnosis or therapeutic treatment of SLE.

### CROSS-REFERENCE TO RELATED APPLICATIONS

This patent application claims the benefit of priority from European Patent Application No. 09167004.2, filed Jul. 31, 2009, the contents of which are incorporated herein by reference.

### FIELD OF INVENTION

The present invention refers to methods for the identification of diagnostic antibodies in auto-immune diseases such as Systemic Lupus Erythematosus (SLE), and therefore useful tools for diagnosis or therapeutic treatment of SLE.

### BACKGROUND

Systemic lupus erythematosus (SLE) is an auto-immune disease characterized by the presence of antibodies against nuclear components and against ribosomal proteins. It has been shown that antibodies against three protein components of the 60S ribosomal subunit, called P0, P1, and P2, are specifically associated with SLE and more frequently detected in active disease, especially in patients with nephritis [K. B. Elkon et al., J. Exp. Med. 1985, 162, 459; A. M. Francoeur et al., J. Immunol. 1985, 135, 2378; E. Bonfa et al., N. Engl. J. Med. 1987, 317, 265]. The anti-ribosomal P antibodies were identified in sera from SLE patients by immunoblotting analysis on ribosomal extracts.

It has been verified that the antigenic determinant of ribosomal proteins is located in the C-terminal region of the ribosomal protein eL12 of the organism Artemia Salina [K. Elkon et al., Proc. Nat. Acad. Sci. USA 1986, 83, 7419]. A method based on the use of a peptide comprising the last 22 amino acids of ribosomal P proteins (C-22: NH₂-Lys-Lys-Glu-Glu-Lys-Lys-Glu-Glu-Ser-Glu-Glu-Glu-Asp-Glu-Asp-Met-Gly-Phe-Gly-Leu-Phe-Asp-COOH, SEQ ID NO: 1) [N. Brot et al., US4865970] has been demonstrated to detect SLE specific auto-antibodies in about 10-20% of patients sera [K. Elkon et al., Proc. Nat. Acad. Sci. USA 1988, 85, 5186; F. B. Karassa et al., Arthritis Rheum. 2006, 54, 312]. The sensitivity of the ELISA based on the peptide C-22 was further improved using the four-branched multiple antigenic peptide (MAP) of C-22 [B. F. Bruner et al., Ann. N.Y. Acad. Sci. 2005, 1051, 390] or of the last C-terminal 13 amino acids [L. Caponi et al., J. Immunol. Methods 1995, 179, 193]. Nevertheless, some studies have been performed to identify conformational epitopes [E. Koren et al., J. Clin. Invest. 1992, 89, 1236; Mahler et al., Clin. Vaccine Immunol. 2006, 13, 77] or different epitopic regions of ribosomal P proteins [N. Fabien et al., J. Autoimmun. 1999, 13, 103]. A peptide scan through the complete amino acid sequences of human P0, P1, and P2 confirmed the reported immunodominance of the C-terminal epitope [M. Mahler et al., J. Mol. Med. 2003, 81, 194]. No central or N-terminal peptides were demonstrated to be more active than C-22 in the test conditions. A further epitope mapping, performed on the full sequence of human P0 ribosomal protein, demonstrated the presence of other antigenic regions less active than C-22 [B. F. Bruner et al., Ann. N.Y. Acad. Sci. 2005, 1051, 390].

### SUMMARY OF THE INVENTION

A technical problem still unsolved by the above prior art is the provision of specific and reliable diagnostic tools for auto-immune diseases, specifically SLE.

The present invention refers to the use of peptides derived from the N-terminal portion of the P1 ribosomal protein, that surprisingly detect specific auto-antibodies in SLE patients sera. In addition, another embodiment of the present invention is the development of a more sensitive peptide-based method of detecting SLE. The peptides of the present invention, also conjugated to a resin, can be used for therapeutic treatments of SLE patients.

Authors performed a complete epitope mapping of the sequences of human P1 and P2 proteins. 14 peptides of 20 amino acids were synthesized with an overlapping of 5 amino acids comprising the N-terminal regions. The peptides were obtained as pure products (purity >95%) and were tested on 25 sera of SLE patients and 68 healthy blood donors.

It has been surprisingly found, and is a subject of the present invention, that a peptide derived from the N-terminal region of ribosomal P1 protein, of 10-20 amino acids, has a very efficient role in the recognition of auto-antibodies typical of auto-immune diseases and is therefore a useful tool for diagnosis or therapeutic treatment of SLE.

It is an object of the invention to provide a multimeric branched peptide comprising:
a multiple antigenic peptide (MAP) nucleus structure; and
a linear peptide having an amino acid sequence of at least 10 consecutive amino acids comprised in the sequence of SEQ ID NO: 2, bonded through a carbamido link to each of amino terminal residues of the MAP nucleus structure, wherein the linear peptides are the same as, i.e. equal to or different from each other.

In one aspect of the present invention, there are provided multimeric branched peptides comprising:

a multiple antigenic peptide (MAP) core; and

a peptide having an amino acid sequence of at least 10 consecutive amino acids of SEQ ID NO: 2,

wherein the peptide is covalently attached to the MAP core.

In one preferred aspect, the present invention provides multimeric branched peptides comprising:

a multiple antigenic peptide (MAP) core; and

at least two or more peptides (e.g., up to 16 peptides such as 2, 3, 4, 5, 6, 7, 8) each having an amino acid sequence of at least 10 consecutive amino acids (e.g., 10, 11, 12, 13, 14, 15, 16, 17, 18, 19, 20) of SEQ ID NO: 2,

wherein the MAP core contains at least two or more amine terminals (e.g., up to 16 amine terminals such as 2, 4, 8, 16), and each of the peptides are covalently attached to the amine terminals of the MAP core.

In another preferred aspect, there are provided multimeric branched peptides in which each of the peptides are attached to the amine terminals of the MAP core forming a urea linkage (carbamido linkage). Alternatively, each of the peptides are attached to the amine terminals of the MAP core forming an amide linkage.

In yet another preferred aspect, the MAP core has a branched peptide structure.

According to the invention, a MAP nucleus structure (core) is:

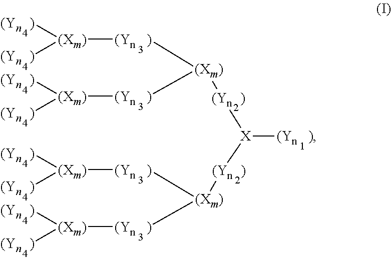

wherein X is an amino acid having at least two amino functional residues (e.g., lysine, ornithine, or 2,3-diaminopropionic acid), Y is an amino acid selected from the group of beta-alanine, alanine, glycine or 6-aminocaproic acid, m is 0 or 1, n₁ n₂ n₃ n₄ are integer numbers comprised between 0 and 10, and wherein bonds are amide bonds.

In one preferred aspect, the MAP core useful for the present invention has the structure:

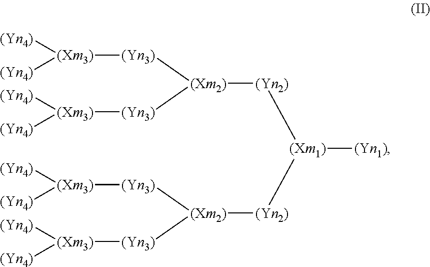

wherein

X, in each occurrence, is independently an amino acid having at least two amino functional residues (e.g., lysine, ornithine, or 2,3-diaminopropionic acid);

Y, in each occurrence, is independently an amino acid (e.g., beta-alanine, alanine, glycine or 6-aminocaproic acid);

m₁ is 1;

m₂ and m₃ are independently 0 or 1; and

n₁, n₂, n₃ and n₄ are independently zero or positive integers of from about 1 to about 10 (e.g., 1, 2, 3, 4, 5, 6), preferably zero or positive integers of from about 1 to about 3 (e.g., 1, 2, 3).

In one preferred embodiment, m₂ is 1, and m₃ is 0 or 1.

In one embodiment, m₂ is 1, and m₃ is 0. Alternatively, m₂ and m₃ are both 1.

In another embodiment, m₂ is 0, when m₃ is 0.

In another preferred embodiment, X is lysine.

For purposes of the present invention, the amino acids defined as Y are the same or different when n₁, n₂, n₃ and n₄ are equal to 2 or greater than 2.

In certain aspects, Y can be a peptide when n₁, n₂, n₃ and n₄ are equal to 2 or greater than 2. The peptide can range in size, for instance, from about 2 to about 10 amino acids (e.g., 2, 3, 4, 5, or 6).

In one preferred embodiment, n₁ is 1.

In another preferred embodiment, n₂ is 0.

In yet another preferred embodiment, n₃ is 0.

In yet another preferred embodiment, n₄ is 0.

In yet another preferred embodiment, n₂, n₃ and n₄ are all zero.

In yet another preferred embodiment, (Yn₁) is beta-alanine where n₁ is 1.

In some preferred embodiments, the multimeric branched peptides described herein include a tetrameric MAP core. In this regard, n₄ is zero. Preferably, n₂, n₃ and n₄ are all zero.

For purposes of the present invention, the amino acid can be selected from any of the known naturally-occurring L-amino acids, e.g., alanine, valine, leucine, isoleucine, glycine, serine, threonine, methionine, cysteine, phenylalanine, tyrosine, tryptophan, aspartic acid, glutamic acid, lysine, arginine, histidine, proline, and/or a combination thereof, to name but a few.

In alternative aspects, derivatives and analogs of the naturally occurring amino acids, as well as various art-known non-naturally occurring amino acids (D or L), hydrophobic or non-hydrophobic, are also contemplated. Simply by way of example, amino acid analogs and derivates include: 2-aminoadipic acid, 3-aminoadipic acid, beta-alanine, beta-aminopropionic acid, 2-aminobutyric acid, 4-aminobutyric acid, piperidinic acid, 6-aminocaproic acid, 2-aminoheptanoic acid, 2-aminoisobutyric acid, 3-aminoisobutyric acid, 2-aminopimelic acid, 2,4-aminobutyric acid, desmosine, 2,2-diaminopimelic acid, 2,3-diaminopropionic acid, N-ethylglycine, N-ethylasparagine, 3-hydroxyproline, 4-hydroxyproline, isodesmosine, allo-isoleucine, N-methylglycine or sarcosine, N-methyl-isoleucine, N-methyllysine, N-methylvaline, norvaline, norleucine, ornithine, and others, that are listed in 63 Fed. Reg., 29620, 29622, incorporated by reference herein.

According to the present invention, the MAP core includes, but is not limited to:

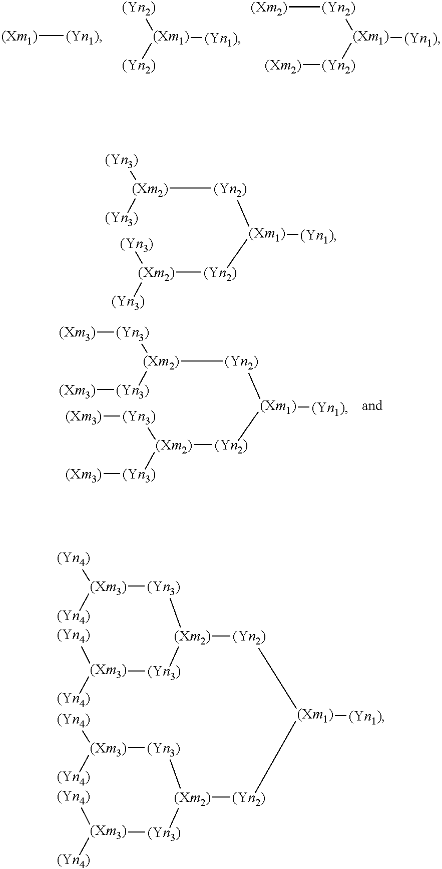

wherein,

X, in each occurrence, is independently an amino acid having at least two amino functional residues (e.g., lysine);

Y, in each occurrence, is independently an amino acid;

m₁, m₂ and m₃, in each occurrence, are all 1; and

n₁, n₂, n₃ and n₄ are independently zero or positive integers of from about 1 to about 10 (e.g, 1, 2, 3, 4, 5, 6), preferably zero or positive integers of from about 1 to about 3 (e.g., 1, 2, 3).

According to the present invention, the MAP cores are substantially non-antigenic. For purposes of the present invention, “effectively non-antigenic”, “substantially non-antigenic” or “substantially immunologically inert” MAP cores refer to MAP core molecules understood in the art as not eliciting an appreciable immune response by auto-immune antibodies present in patients with auto-immune disease (preferably SLE). The MAP cores do not elicit an appreciable immune response by antibodies against the ribosomal proteins (P0, P1, P2) present in SLE patients.

An illustrative example of such MAP cores includes

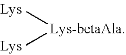

In this regard, Xm₁ and Xm₂ are all lysine, Yn₁ is beta-alanine, and n₂ is zero.

As it is known from the scientific literature [J. P. Tam, Proc. Natl. Acad. Sci. USA 1988, 85, 5409], the MAP structure is characterized by an immunological inert amino acid core, generally a branched core of lysine or of lysine and beta-alanine, to which a number of identical copies of the antigen sequence of interest, generally 4 or 8 copies, are bound. The expression “immunological inert core” means that, when MAPs are used as immunogens, they don't induce the production of antibodies directed against the central amino acid core.

Preferably the multimeric branched peptide is a tetrameric branched peptide in which each of the linear peptides have an amino acid sequence belonging to the following group: amino acids sequence 1-20 of SEQ ID NO: 2; amino acids sequence 1-10 of SEQ ID NO: 2 (SEQ ID NO:3); amino acids sequence 6-16 of SEQ ID NO: 2 (SEQ ID NO: 4); amino acids sequence of 11-20 of SEQ ID NO: 2 (SEQ ID NO: 5), as in the below scheme:

| | | |
|---|---|---|
| 1 | SEQ ID NO: 2 | Leu-Ile-Lys-Ala-Ala-Gly-Val-Asn-Val- |
| | | Glu-Pro-Phe-Trp-Pro-Gly-Leu-Phe-Ala- |
| | | Lys-Ala |
| 2 | aa 1-10 | Leu-Ile-Lys-Ala-Ala-Gly-Val-Asn-Val- |
| | SEQ ID NO: 3 | Glu |
| 3 | aa 6-16 | Gly-Val-Asn-Val-Glu-Pro-Phe-Trp-Pro- |
| | SEQ ID NO: 4 | Gly-Leu |
| 4 | aa 11-20 | Pro-Phe-Trp-Pro-Gly-Leu-Phe-Ala-Lys- |
| | SEQ ID NO: 5 | Ala |

According to the present invention, there are provided the multimeric branched peptides comprising:

(a) a multiple antigenic peptide (MAP) core having the structure:

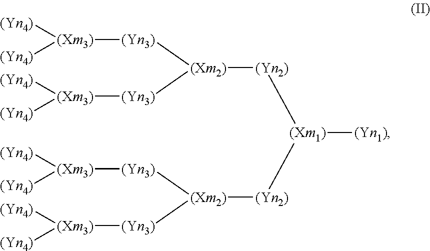

wherein

X, in each occurrence, is independently an amino acid having at least two amino functional residues (e.g., lysine);

Y, in each occurrence, is independently an amino acid;

m₁ is 1;

m₂ and m₃ are independently 0 or 1; and

n₁, n₂, n₃ and n₄ are independently zero or positive integers of from about 1 to about 10 (e.g, 1, 2, 3, 4, 5, 6), preferably zero or positive integers of from about 1 to about 3 (e.g., 1, 2, 3); and

(b) at least two or more peptides (e.g., up to 16 peptides such as 2, 3, 4, 5, 6, 7, 8) each having an amino acid sequence of at least 10 consecutive amino acids (e.g., 10, 11, 12, 13, 14, 15, 16, 17, 18, 19, 20) set forth in SEQ ID NO: 2,

wherein the MAP core contains at least two or more amine terminals (e.g., up to 16 amine terminals such as 2, 4, 8), and each of the peptides are covalently attached to the amine terminals of the MAP core.

Preferably, the tetrameric branched peptide comprises four identical copies of the above reported preferred peptides. The following tetrameric branched peptides containing the amino acid sequences as above are preferred:

| | |
|---|---|
| 5 | 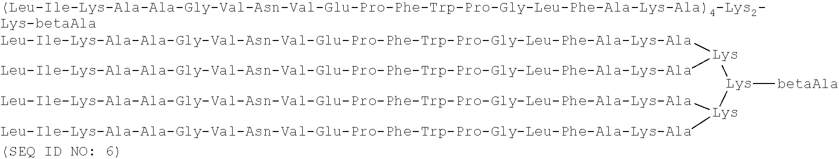 |
| 6 | 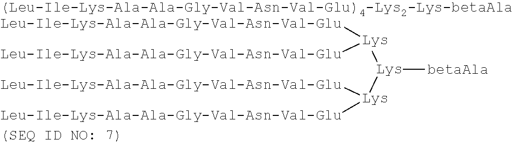 |
| 7 | 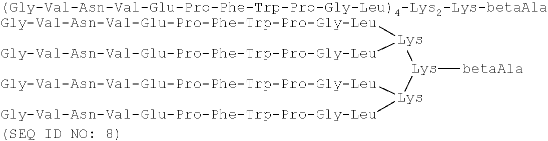 |
| 8 | 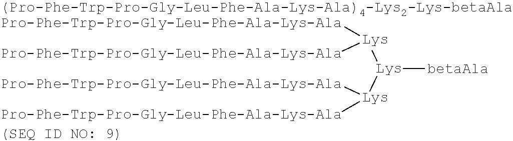 |

It is another object of the invention to provide a peptide composition comprising at least one multimeric branched peptide as above disclosed and a further tetrameric branched peptide comprising a MAP nucleus structure and four linear peptides having the amino acid sequence 10-22 of SEQ ID NO: 1.

Another aspect of the present invention is the development of a diagnostic method to increase the specificity of the immunological assays. The preferred diagnostic method is performed testing the peptides 1-8 and the four-branched MAP of the last C-terminal 13 amino acids of the ribosomal protein eL12 of the organism Artemia Salina (C-terminus 13 amino acids sequence of SEQ ID NO: 1: Glu-Glu-Glu-Asp-Glu-Asp-Met-Gly-Phe-Gly-Leu-Phe-Asp: SEQ ID NO: 10).

The multimeric branched peptide in the MAP format has the following structure:

| | |
|---|---|
| 9 | 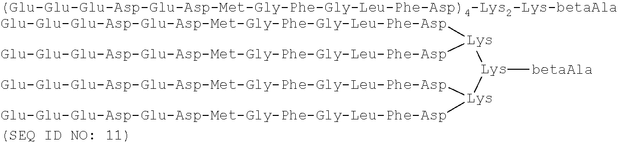 |

The combined use of the two different antigenic regions allowed to detect the presence and/or measuring of the levels of anti-ribosomal P protein antibodies in samples of biological fluids in an increased number of SLE patients.

The immunological assay based on the multimeric branched peptides described herein further include the preferred peptides comprised in SEQ ID NO: 1 (e.g., the tetrameric MAP branched of peptide formula 9) and can be performed either by mixing them (e.g., peptide #9 and one of peptides #5-8 or combinations thereof) during the coating step or using them in parallel, as in multiplexed experiments.

It is another object of the invention to provide the use of a peptide comprising an amino acid sequence of at least 10 consecutive amino acids sequence of SEQ ID NO: 2, for the detection of auto-antibodies specific for an auto-immune disease. Preferably said peptide has an amino acid sequence belonging to the following group: amino acids 1-20 of SEQ ID NO: 2; amino acids 1-10 of SEQ ID NO: 2; amino acids 6-16 of SEQ ID NO: 2; amino acids 11-20 of SEQ ID NO: 2. The peptides of the inventions may be bound to a solid support.

It is another object of the invention to provide an immunodiagnostic kit for detecting auto-antibodies specific for an auto-immune disease comprising as selective reagent the peptide or the composition as above disclosed. Preferably the auto-immune disease is SLE.

It is another object of the invention to provide a method for detecting auto-antibodies specific for an auto-immune disease in a fluid sample comprising the step of incubating said fluid sample with the peptide or the composition as above disclosed, and detecting bound antibodies. Preferably the auto-immune disease is SLE.

According to the present invention, the method for detecting the presence of auto-immune antibodies against ribosomal proteins in mammals includes contacting a fluid sample (e.g., serum, plasma) with a multimeric branched peptide described herein.

In one embodiment, the multimeric branched peptide useful in the method includes any one of peptides #5-8 or combinations thereof.

In a further embodiment, the method uses the multimeric branched peptide described herein in combination with C-terminus 13 amino acids of SEQ ID NO: 1.

In an alternative and further aspect, the method for detecting the presence of auto-immune antibodies against ribosomal proteins in mammals includes contacting a fluid sample with a peptide containing an amino acids sequence of at least 10 consecutive amino acids (e.g., 10, 11, 12, 13, 14, 15, 16, 17, 18, 19, 20) of SEQ ID NO: 2.

In order to perform the immunological assay, the peptides can be adsorbed or covalently linked or modified with a carrier to bind it to a solid support (e.g. chips, microspheres, gold, polystyrene, reactor vessels or wells, micro-titre plate). In a first step of the assay method, the sample of biological fluid to be analyzed is placed in contact and incubated with the preferred peptide linked on the solid support. Any anti-ribosomal P protein antibodies that are possibly present in the sample are thus specifically bound to the preferred peptide, producing an antigen/antibody complex. The anti-ribosomal P protein antibodies to be detected in the immunoassay are IgG immunoglobulins. The evaluation of the presence and the quantization of the antigen/antibody complex can be performed with a spectroscopic, a piezoelectric, or an electrochemical biosensor.

In a preferred embodiment, the above described method is an ELISA immunological assay in which an indicator antibody, like an anti-human IgG, is conjugated to an enzyme and is added to measure the antibody titer by a spectroscopic transducer.

In another preferred embodiment of the present invention, peptides as above disclosed in free form or bound to appropriate resins, can be used for the treatment of patients affected by SLE, since, thanks to their high specificity of antibody recognition, they can be used to achieving selective antibody removal and also immunomodulation of the disease.

### DETAILED DESCRIPTION OF THE INVENTION

Examples disclosing the preparation of some peptides according to the invention, are provided in the following examples for illustrative, non limiting purposes of the invention.

### Example 1

### Peptide Synthesis

Peptide sequences were selected after an epitope mapping was performed on the full sequence of human ribosomal P1 and P2 proteins. Peptides were synthetized using a Wang resin preloaded with the C-terminal amino acid of the sequence or with the MAP core and following the Fmoc/tBu solid-phase peptide strategy. Fmoc deprotections were carried out in 20 min with 20% piperidine in DMF. Coupling reactions were performed by treating the resin for 45 min with a 0.5 M solution of the Fmoc-protected amino acids and HOBt in DMF (2.5 equiv), a 0.5 M solution of TBTU in DMF (2.5 equiv), and 4 M NMM in DMF (5 equiv). Peptide cleavage from the resin and deprotection of the amino acid side chains were carried out in 3 h with TFA/thioanisole/ethanedithiol/phenol/H₂O (82.5:5:2.5:5:5). The crude products were precipitated with cold Et₂O, centrifuged, and lyophilized. The pure peptides were obtained by HPLC in a purity>95% and characterized by mass spectrometry (ESI-Orbitrap and/or MALDI-TOF).

### Example 2

### Antibody Detection by ELISA

A peptide, prepared as described in example 1 and diluted in 0.05 M carbonate buffer (pH 9.6) or in PBS buffer (pH 7.4) was adsorbed onto 96-wells microtitre plates for 4 h at r.t. The wells were treated for 1 h at r.t. with FBS (FBS 5% in PBS). The patients serum samples (diluted 1:100) were then loaded onto the plate and allowed to react overnight at +4° C. After plate washing, an anti human-IgG antibody conjugated to the enzyme alkaline phosphatase was added and incubated for 3 h at r.t. After washings, the alkaline phosphate substrate p-nitrophenyl phosphate was added to the wells. After the quenching of the enzymatic reaction with 1 M NaOH, the absorbance was evaluated at a wavelength of 405 nm.

### Example 3

### ELISA Based on the Contemporary Use of Peptides 1 and 9

To achieve a highly sensitive immunoassay, an equimolar mixture of peptides 1 and 9 were allowed to adsorb to 96-wells microtitre plates for 4 h at r.t. The test was then performed as described in example 2.

### Results

In total, 25 serum samples of patients suffering from SLE and 68 serum of healthy blood donors were tested by this method. Results are reported in Table 1.

TABLE 1
ELISA results of peptides 1, 5 and
9 with SLE and healthy donor sera.

| Peptide | SLE positive sera | healthy donor positive sera |
|---|---|---|
| 1 | 8/25 (32%) | 1/68 (1%) |
| 5 | 4/25 (16%) | 5/68 (7%) |
| 9 | 9/25 (36%) | 1/68 (1%) |

Considering that anti-ribosomal P antibodies are present in 10-20% of SLE patients sera, as object of this patent, peptides recognizing more than 10-20% of SLE patients sera were preferred.

Peptide 1 of formula (I) was able to recognize antibodies in 32% of SLE patients and in only 1% of healthy controls. Peptide 1 and 9 recognized different populations of auto-antibodies in SLE sera and the total number of SLE sera positive to peptide 1 and 9 is 17/25 (68%). In particular, among the 13 SLE sera positive to peptide 9, only 4 of serum showed a cross-reactivity with peptide 1.

Using the method based on peptide 1 it was possible to detect anti ribosomal antibodies in 3 SLE serum negative to peptide 9.

The results demonstrated the presence of two different subclasses of auto-antibodies: one reactive against an N-terminal domain of human P1, and the other one positive to the C-terminal region common to all the three ribosomal P proteins.

The method set up evaluating the SLE sera positive to peptide 1 and 9 has a sensitivity of 68%.

### Example 4

### Antibody Detection by Covalent ELISA

A peptide, prepared as described in example 1, was covalently linked to a 96-wells microtitre plate (carboxy or amine binding plate) using sulfo-N-hydroxysuccinimide and 1-ethyl-3-(3-dimethylaminopropyl)carbodiimide. Peptide coupling took place for 2 h at r.t. The plate was blocked for 1 h with 5% FBS in PBS or with 0.5 M Gly (pH 8). The patients serum samples were diluted 1:100 in FBS buffer and were then loaded onto the plate for 1.5 h at r.t. After plate washings, an anti human-IgG antibody conjugated to the enzyme alkaline phosphatase was added and incubated for 1.5 h at r.t. After washings, the alkaline phosphate substrate p-nitrophenyl phosphate was added to the wells. After the quenching of the enzymatic reaction with 1 M NaOH, the absorbance was evaluated at a wavelength of 405 nm.

### Results

Results for the antibody detection by covalent ELISA for peptides 1-4 are reported in Table 2.

TABLE 2
ELISA results of peptides 1-4 with SLE and healthy donor sera.

| Peptide | SLE positive sera | healthy donor positive sera |
|---|---|---|
| 1 | 4/8 (50%) | 0/6 (0%) |
| 2 | 3/8 (37%) | 0/6 (0%) |
| 3 | 1/8 (12%) | 0/6 (0%) |
| 4 | 1/8 (12%) | 0/6 (0%) |

### Example 5

### Conjugation of a Peptide to Resin for Therapeutic Uses

A peptide prepared as described in example 1 was conjugated to sepharose resin preactivated with CNBr, according to the usual reaction protocols advised by the manufacturers to obtain a resin-peptide conjugate. The product thus obtained is useful as for example for the preparation of plates for the diagnosis or treatment of patients affected by SLE.

1. A multimeric branched peptide comprising;
a multiple antigenic peptide (MAP) core; and
a peptide having an amino acid sequence of at least 10 consecutive amino acids of SEQ ID NO: 2,
wherein the peptide is covalently attached to the MAP core.

2. The multimeric branched peptide of claim 1, wherein the MAP core contains at least two or more amine terminals, at least two or more peptides each have an amino acid sequence of at least 10 consecutive amino acids of SEQ ID NO: 2, and each of the peptides are covalently attached to the amine terminals of the MAP core.

3. The multimeric branched peptide of claim 1, comprising
a MAP nucleus structure; and
a linear peptide having an amino acid sequence of at least 10 consecutive amino acids sequence of SEQ ID NO: 2, bonded through a carbamido link to each of amino terminal residues of the MAP nucleus structure, wherein the linear peptides are equal or different each others.

4. The multimeric branched peptide according to claim 1 being a tetrameric branched peptide and having the amino acid sequence of the linear peptides belonging to the following group: amino acids sequence 1-20 of SEQ ID NO: 2; amino acids sequence 1-10 of SEQ ID NO: 2; amino acids sequence 6-16 of SEQ ID NO: 2; and amino acids sequence 11-20 of SEQ ID NO: 2.

5. A peptide composition comprising at least one multimeric branched peptide of claim 1 and a tetrameric branched peptide comprising a MAP nucleus structure and four linear peptides having the amino acid sequence 10-22 of SEQ ID NO: 1.

6. The multimeric branched peptide according to claim 1, wherein the multimeric branched peptide is bound to a solid support.

7. An immunodiagnostic kit for detecting auto-antibodies specific for an auto-immune disease comprising a multimeric branched peptide of claim 1 or a composition containing a multimeric branched peptide of claim 1.

8. The immunodiagnostic kit according to claim 7 wherein the auto-immune disease is SLE.

9. A method for detecting auto-antibodies specific for an auto-immune disease in a fluid sample comprising the step of incubating said fluid sample with a multimeric branched peptide of claim 1 or a composition containing a multimeric branched peptide of claim 1, and detecting bound antibodies.

10. The method for detecting auto-antibodies according to claim 9 wherein the auto-immune disease is SLE.

11. A method for detecting the presence of auto-immune antibodies against ribosomal proteins in mammals comprising contacting a fluid sample with a multimeric branched peptide of claim 1.

12. A method for detecting the presence of auto-immune antibodies against ribosomal proteins in mammals comprising contacting a fluid sample with a peptide containing an amino acid sequence of at least 10 consecutive amino acids of SEQ ID NO: 2.

13. A multimeric branched peptide according to claim 1 for medical use.
